(19) United States
(12) Patent Application Publication
(10) Pub. No.: US 2021/0212911 A1
(43) Pub. Date: Jul. 15, 2021

(54) METHOD FOR PROMOTING HOMEOSTASIS IN THE SKIN

(71) Applicant: Etho Holdings, Inc., La Jolla, CA (US)

(72) Inventor: Erica Oberg, La Jolla, CA (US)

(73) Assignee: Etho Holdings, Inc., La Jolla, CA (US)

(21) Appl. No.: 17/146,356

(22) Filed: Jan. 11, 2021

Related U.S. Application Data
(60) Provisional application No. 62/958,718, filed on Jan. 9, 2020.

(51) Int. Cl. A61K 8/34; A61K 8/67; A61K 8/9789; A61K 8/9711; A61K 8/49; A61K 8/31; A61K 8/73; A61K 8/44; A61Q 19/00

(52) U.S. Cl. CPC A61K 8/347; A61K 8/675; A61K 8/9789; A61K 8/9711; A61K 8/4973; A61K 2800/10; A61K 8/676; A61K 8/735; A61K 8/447; A61Q 19/00; A61K 8/31

(57) ABSTRACT

The new product utilizes a novel combination of cannabidiol and niacinamide to maintain skin homeostasis through synergistic pathways which help to protect the skin of an individual.

This patent application claims priority to U.S. Provisional Patent Application No. 62/958,718 filed on Jan. 9, 2020, which is incorporated by reference herein, in its entirety.

### BACKGROUND OF THE INVENTION

The present invention relates to promoting homeostasis in human skin through the novel synergistic combination of niacinamide and cannabidiol.

Healthy, youthful skin is defined by its ability to maintain homeostasis. Skin is the largest organ in the human body, responsible for protecting the delicate inner functions of the body from the external environment, as well as participating in hormone activation and immune regulation. The epidermal cells have to maintain strong cell membranes with the appropriate fatty acid composition. The inner layers must be supplied with antioxidants and appropriate nutrients to ensure optimal cell replication, which involves DNA repair secondary to any oxidative stress such as UV or toxin exposure. Melanocytes and immune cells are also in this surface layer. A strong epidermal layer ensures a strong physical barrier to environmental exposures that cause oxidative stress. Importantly, vitamin D production also begins here. The dermis contains sweat glands, sebaceous glands, nerve endings, and more specialized immune mediators/cytokines. Dysregulation (through oxidative stress) at the level of the dermis results in a number of skin conditions ranging from mild to severe—acne and wrinkling to bullous pemphigoid and cancer.

To maintain healthy skin, the normal functions of the skin must remain in balance. Overstimulation can result in inflammation, irritation, overly rapid cell turnover and desquamation. Underactivity results in atrophy, dehydration, and degeneration. This is called maintaining homeostasis—the sweet spot of appropriate functionality that is adaptive and responsive to the changing environmental conditions.

A number of natural agents are known to promote homeostasis. Certain botanical extracts are particularly renowned for these properties and are term adaptogens. Many adaptogens work as weak agonists. That is, the bind weakly to receptors (for example, receptors on immune cells) and if that receptor is underactive, it stimulates activity. If the receptors are overactive and flooded with strong agonists, the adaptogen displaces some of the overstimulation by weakly binding, thus downregulating the effect. Common adaptogens include ginseng (Panax spp), Rhodiola rosea, ashwaganda (Withania somnifera), and cannabidiol (Cannabis sativa constituent). Adaptogens are one potent strategy to maintain healthy skin homeostasis.

Niacinamide contributes to energy homeostasis in part related to methyl-donation and in part related to preventing the depletion of cellular nicotinamide adenine dinucleotide (NAD) levels. Niacinamide prevents the depletion of cellular NAD through its status as an NAD precursor, and its interactions with poly-ADP-ribose polymerase-1 (PARP-1). When human skin cells are exposed to UV-radiation in laboratory settings, the addition of Niacinamide promotes DNA repair activity, and reverses DNA damage in NAD-depleted cell cultures. Niacinamide also promotes energy homeostasis through the methyl-donation redox reactions of NADH and NADPH

The combination of cannabidiol and niacinamide represent a novel invention because the combination has synergistic properties representing a broad spectrum invention capable of promoting skin homeostasis through both relevant pathways.

### SUMMARY

The new product utilizes a novel combination of cannabidiol and niacinamide to maintain skin homeostasis through synergistic pathways which help to protect the skin of an individual.

### BRIEF DESCRIPTION OF THE DRAWINGS

FIG. 1 is a diagram depicting several of the relevant cellular mechanisms and pathways of niacinamide.

FIG. 2 is a diagram depicting some of the relevant cellular mechanisms and pathways of cannabidiol.

FIG. 3 is a diagram depicting some of the relevant pathways of cannabidiol's anti-inflammatory effects.

### DETAILED DESCRIPTION OF THE INVENTION

The invention relates to a skin care product comprising of a mixture of cannabidiol and niacinamide. The skin care product may be a type of sunscreen, moisturizer, lotion, serum, topical oil, balm, cream or other kind of skin care product known in the art. The invention is described herein.

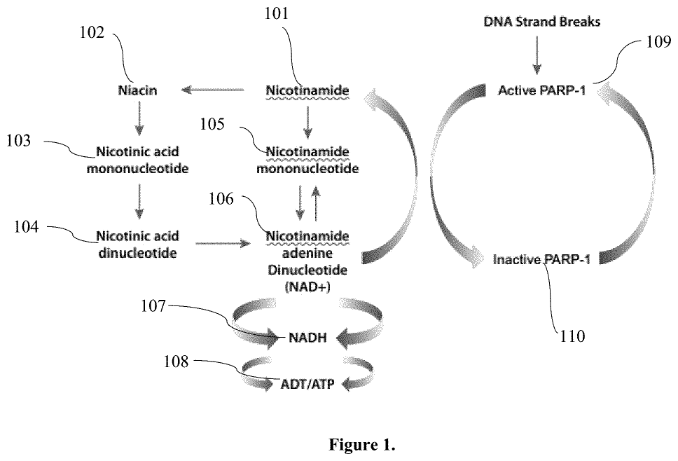

One embodiment of the product contains a mixture of 5%-25% niacinamide and 3%-25% of cannabidiol. As seen in FIG. 1, a solution containing 5%-25% niacinamide operates within specific pathways of the cell. Niacinamide 101 can be metabolized into Niacin 102 or Nicotinamide mononucleotide 105. Niacin 102 is eventually metabolized into Nicotinamide adenine dinucleotide 106 after being metabolized to Nicotinic acid mononucleotide 103 and Nicotinic acid dinucleotide 104. Nicotinamide mononucleotide 105 is metabolized into Nicotinamde adenine dinucleotide 106. Once Niacinamide 101 is converted to Nicotinamide adenine dinucleotide 106, it can be converted to ATP 108 through the NADH pathway 107 or it can be metabolized to Niacinamide 101 through the action of Active PARP-1 109. If Nicotinamide adenine dinucleotide 106 is metabolized by Active PARP-1 109, Niacinamide 101 is formed and Inactive PARP-1 110 is formed.

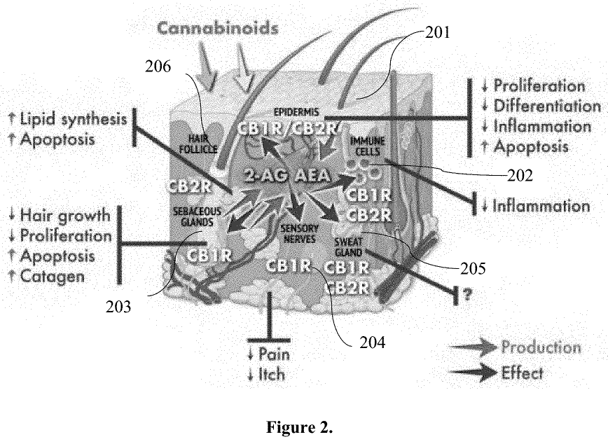

As seen in FIG. 2, a topical solution containing 3%-25% cannabidiol interacts with cannabinoid receptor 1 (CB1R) and cannabinoid receptor 2 (CB2R). CB1R is known to be present in the epidermis 201, immune cells 202, sebaceous glands 203, sensory nerves 204 and sweat glands 205. CB12R is known to be present in the epidermis 201, hair follicle 206, immune cells 202 and sweat glands 205. CB2R activation has been known to increase DNA methylation in human keratinocytes, and CB1R and CB2R activation has been known to reduce inflammation in Allergic Contact Dermatitis, epidermolysis bullosa, eczema, acne vulgaris, scarring & keloid formation, seborrheic conditions, melanoma, and other skin conditions.

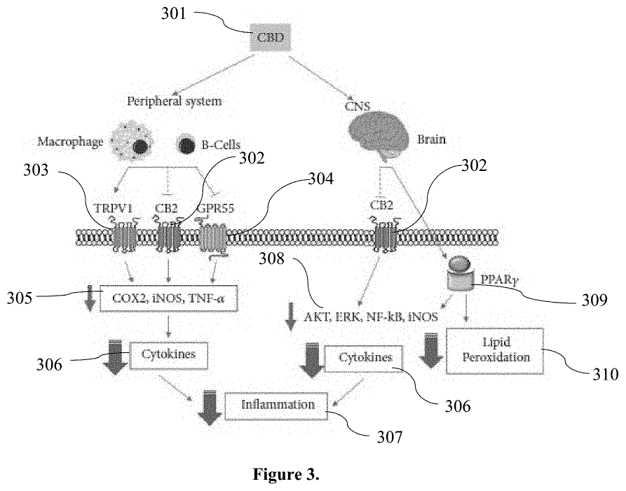

Examples of the pathways used to reduce inflammation in these conditions can be seen in FIG. 3, where cannabidiol 301 interacts with CB2R 302, TRPV1 303 and GPR55 304 to decrease the downstream production of prostaglandins, reactive oxygen species and cytokines 306 in the cell, which reduces peripheral inflammation 307. It does this by decreasing cyclooxygenase 2 activity, inducible nitric oxide synthase and tumor necrosis factor alpha 305. Cannabidiol 301 also reduces inflammation 307 by inhibiting MAPK and downregulating NF-kB 308 along with PPARγ 309 mediated reduction of lipid peroxidation 310.

Additional nutrients and botanicals that promote homeostasis include, but are not limited to, Camellia sinensis, Laminaria japonica, fucoxantin, lycopene, zeaxanthin, ascorbic acid, amino acids, riboflavin, green tea extract, hyaluronic acid, glutathione and others. A solution containing 0.1% of any of these nutrients and botanicals may promote homeostasis. Sufficient cofactors and metabolic intermediaries must be present at appropriate concentrations to ensure healthy cellular respiration though the Krebs cycle and ATP production. Without such nutrient bioavailability, homeostatic mechanisms are compromised.

Because niacinamide and cannabidiol are metabolized and utilized in different cellular mechanisms and pathways, a solution containing both niacinamide and cannabidiol allows skin cells to have an improved inflammatory response, improved immunity response, improved oxidative stress repair, improved DNA repair, and improved cellular energy production and cell turnover.

While certain preferred embodiments of the invention have been specifically disclosed, it should be understood that the invention is not limited thereto as many variations will be readily apparent to those skilled in the art and the invention is to be given its broadest possible interpretation within the terms of the following claims. Thus a skin care product comprising a mixture of cannabidiol and niacinamide has been described.

What is claimed is:

1. A skin care product comprising:
at least 3% of cannabidol;
at least 5% of niacinamide.

2. The skin care product of claim 1 wherein the cannabidol content is 10%.

3. The skin care product of claim 1 wherein the maximum cannabidol content is 25% and the maximum niacinamide content is 25%.

4. The skin care product of claim 1 further comprising:
At least 0.1% of Camellia sinensis.

5. The skin care product of claim 1 further comprising:
At least 0.1% of Laminaria japonica.

6. The skin care product of claim 1 further comprising:
At least 0.1% of fucoxantin.

7. The skin care product of claim 1 further comprising:
At least 0.1% of lycopene.

8. The skin care product of claim 1 further comprising:
At least 0.1% of zeaxanthin.

9. The skin care product of claim 1 further comprising:
At least 0.1% of ascorbic acid.

11. The skin care product of claim 1 further comprising:
At least 0.1% riboflavin.

12. The skin care product of claim 1 further comprising:
At least 0.1% of green tea extract.

13. The skin care product of claim 1 further comprising:
At least 0.1% of hyaluronic acid.

14. The skin care product of claim 1 further comprising:
At least 0.1% glutathione.
